(19) United States
(12) Patent Application Publication
(10) Pub. No.: US 2026/0357188 A1
(43) Pub. Date: Nov. 12, 2026

(54) METHODS AND SYSTEMS FOR ENGINEERING BIOLOGICAL FORMULATIONS

(71) Applicant: Qatch Technologies, LLC, Durham, NC (US)

(72) Inventors: Zehra PARLAK DINC, Chapel Hill, NC (US); Paul Edward MACNICHOL, Raleigh, NC (US)

(21) Appl. No.: 19/653,710

(22) Filed: Apr. 21, 2026

Related U.S. Application Data
(60) Provisional application No. 63/792,417, filed on May 6, 2025.
(60) Provisional application No. 63/806,949, filed on May 16, 2025.

(51) Int. Cl. C12M 1/36; C12M 1/34; G06Q 50/04; G16B 40/10

(52) U.S. Cl. CPC C12M 41/48; C12M 41/26; C12M 41/32; G06Q 50/04; G16B 40/10

(57) ABSTRACT

System and method for building a formulation model with reference to physical properties, including viscosity. The model can be used to guide directed formulation development having beneficial properties. The system and method predict physical properties of formulations having different concentrations, buffer solutions, or other parameters, and identify which tests need to be performed to obtain additional data. A machine learning model, trained on filtered data sets, is utilized in order to make the predictions. The filtering and training methods generate a model having higher predictive power than conventional modeling methods.

### CROSS-REFERENCE TO RELATED APPLICATIONS

This application claims priority to and the benefit of U.S. Provisional Patent Application No. 63/806,949, filed May 16, 2025, and U.S. Provisional Patent Application No. 63/792,417, filed Apr. 22, 2025, each of which is incorporated herein by reference in its entirety.

### TECHNICAL FIELD

The field of the invention relates generally to artificial intelligence-based systems for predicting properties of compound formulations. More specifically, the field of the invention relates to predicting viscosity of different novel formulations and determining which viscosity tests need to be performed.

### BACKGROUND

In order to ensure safety and efficacy of new protein formulations, a suite of tests are required to be performed during development to test for different properties of the formulation, including biological properties such as immunogenicity, as well as physical properties such as elasticity, viscosity, and fluid consistency. Viscosity testing poses a challenge for drug development in terms of cost, time, and equipment, as viscosity of biologic formulations varies at different volumes or with other changes to the formulations, often in difficult to predict ways, as the formulations act non-linearly as non-Newtonian fluids. The viscosity of these formulations differs, for example, based on the concentration of the active compound, the composition of active compound itself, flow velocity, as well as details of the buffer solution used. Viscosity testing, therefore, is not only a single test, but a suite of tests performed on different dose volumes and on formulations with different formulation parameters. Therefore, existing systems as provided to test physical properties, including viscosity, of large numbers of variants of biologic formulations having different formulation parameters.

### SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

In one embodiment, the invention is directed to a system for managing physical measurements of biologic formulations, including a computing device, including a processor and a memory, wherein the processor is configured to execute a plurality of programming instructions that, when operating on the processor, cause the computing device to receive input measurement data of at least one physical property of at least one variant of a biologic formulation, update a preexisting machine learning model using the input measurement data, utilize the updated machine learning model to generate a predicted physical property profile of the biologic formulation, identify at least one selected variant of the biologic formulation for which additional measurement data is suggested, and receive measurement data for at least one additional variant of the biologic formulation, wherein, if the measurement of the at least one additional variant does not match the predicted physical property profile of the biologic formulation, generate an updated predicted physical property profile using the updated machine learning model.

In another embodiment, the invention is directed to a method of managing physical measurements of biologic formulations, including receiving input measurement data of at least one physical property of at least one variant of a biologic formulation, updating a preexisting machine learning model using the input measurement data, utilizing the updated machine learning model to generate a predicted physical property profile of the biologic formulation, identifying at least one selected variant of the biologic formulation for which additional measurement data is suggested, and receiving measurement data for the at least one additional variant of the biologic formulation, wherein, if the measurement of the at least one additional variant does not match the predicted physical property profile of the biologic formulation, generating an updated predicted physical property profile using the updated machine learning model.

In yet another embodiment, the invention is directed to a system for managing rheological testing of biologic formulations, including a computing device, including a processor and a memory, wherein the processor is configured to execute a plurality of programming instructions that, when operating on the processor, cause the computing device to periodically receive population training data including viscosity measurements of various biologic compounds in formulations having various parameters from a federated server to fine-tune a local instance of a machine learning model, receive formulation parameters for a plurality of variants of a biologic formulation, utilize the machine learning model to generate a predicted viscosity profile of the biologic formulation, identify at least one selected variant of the biologic formulation for which additional viscosity measurement data is suggested, receive viscosity measurement data for at least one additional variant of the biologic formulation, and fine tune the machine learning model using the viscosity measurement data for the at least one additional variant of the biologic formulation, wherein, if the measurement of the at least one additional variant does not match the predicted viscosity profile of the biologic formulation, generate an updated predicted viscosity profile using the updated machine learning model.

### BRIEF DESCRIPTION OF THE DRAWINGS

FIG. 1 is a schematic diagram of a system implementing artificial intelligence-based prediction to streamline formulation viscosity testing according to one embodiment of the invention.

FIG. 2 is a schematic diagram of a method for training viscosity prediction models according to one embodiment of the invention.

FIG. 3 is a schematic diagram for a system for predicting rheological properties of a test sample according to one embodiment of the invention.

FIG. 4 is a schematic diagram for a system of generating a predicted viscosity profile based on context input and a target query according to one embodiment of the invention.

### DETAILED DESCRIPTION

The presently disclosed subject matter is described with specificity to meet statutory requirements. However, the description itself is not intended to limit the scope of this patent. Rather, the inventors have contemplated that the claimed invention might also be embodied in other ways, to include different steps or elements similar to the ones described in this document, in conjunction with other present or future technologies.

Biologics, such as monoclonal antibodies (mAbs), pose a number of challenges in formulation development for drug manufacturers. These drugs are usually highly concentrated to enable subcutaneous injection and are difficult to manufacture. Viscosity, in particular, is an important fluid property that needs to be understood by measurement during the formulation development process to ensure that therapeutics can be both manufactured and delivered without an excessive injection force. This concern is especially important when developing high concentration antibody formulations, which can have a broad range of viscosities depending on the specific protein-protein interactions. These formulations can involve high concentration protein formulations greater than 150 mg/mL in some cases. At these high concentrations, a minor change in concentration can lead to a dramatic change in viscosity. Further, the viscosity is often shear-related, necessitating measuring viscosity across a large shear range.

The typical viscosity measurement range depends on the specific application and the type of viscometer used. Generally, for liquid materials, the measurement range is from 0.001 to 1 Pascal-second (1 to 1000 centipoise). The volume of liquid needed for viscosity measurement varies significantly depending on the viscometer type and the specific application but generally ranges from a few milliliters to several hundred milliliters. Rotational viscometers, such as Brookfield viscometers, often require 0.5 mL to 2 mL for cone-plate spindles, while others may need 200-250 mL for straight spindles. In general, it is preferable to have a larger sample volume than the minimum required by the viscometer, especially if repeat measurements are needed, or if the sample needs to be recovered after testing.

Scaling up biologic production involves increasing the volume of manufacturing, often from bench-scale to industrial-scale, while maintaining product quality and purity. This can be achieved through scale-up, where bioreactor size is increased, or scale-out, where multiple bioreactors of the same size are used in parallel. Scale-out offers flexibility and potentially reduces risks associated with scale-up, especially in commercial production. In biologics development, scaling from 96-well plates involves increasing the volume of cell culture media and the number of cells. A biologic manufacturing process typically involves scaling up of production from miniaturized, high-throughput bioreactors (15-250 mL) to bench-scale glass or single-use reactors (1-10 L) and then pilot- and production-scale bioreactors (200-5,000 L or larger).

Thus, biologics development faces a disconnect between the ability to determine viscosity and the volumes of the formulations required. In biologics development, viscosity is often not linear with concentration, especially for concentrated antibody solutions. This non-linear behavior is due to factors such as intermolecular interactions, aggregation, and concentration-dependent fluctuations of antibody regions. Understanding this non-linearity is crucial for formulation development, particularly when aiming for high-concentration formulations for subcutaneous administration. While it is possible to make such a set of viscosity measurements, it is not feasible, in terms of time, money, or equipment, to do so during biological development. What is needed is a system and method for predicting viscosity of a given formulation, such as antibody formulations, across a high shear range, with a minimal amount of material.

The systems and methods disclosed herein utilize a wide range of formulation viscosity data with minimal user input. Using a high shear-rate range viscometer in these viscosity tests enables observing wide shear-rate viscosity with minimal sample volume, thereby identifying developability-related behaviors.

Viscosity profiles for a wide range of shear-rates are measured for multiple proteins with different buffer conditions as basic seed training data. In one embodiment, the basic seed training data includes viscosity profiles for at least one standardized shear-rate for multiple proteins with different conditions measured on a single instrument or a single type of instrument, further including metadata corresponding to the buffer conditions and a unique identifier for the protein. In another embodiment, the basic seed training data includes viscosity measurements and viscosity simulations for at least one standardized shear-rate for multiple proteins in different buffer conditions, further including metadata for the buffer conditions and a unique identifier for the protein.

A machine learning (ML) model is trained on the seed data set to identify major trends related to formulation viscosity, as a generalization. The trained ML model is installed on an on-site computer or stored in one or more cloud servers.

Users with an interest in a new protein or (i.e., a protein not included in the data set) first test the viscosity of a few solutions with the new protein. The number of initial tests can be as small as 1 to 20 or more, depending on material availability and user interest. Data from the initial new protein tests are added to at least one database, either a local database or a cloud-based database. In a preferred embodiment, the data from the initial new protein tests is tagged with at least one unique identifier corresponding to the new protein, where the unique identifier is different from identifiers of any previous proteins in the existing data set. In one embodiment, the protein tests are tagged with at least one of molecular weight, isoelectric point, protein class, hydrodynamic patch, electrostatic charge distribution, and sequence.

The ML model adds the viscosity data generated by these tests for the new protein to the trained set, and updates the data set with the test results being highly weighted and acting as anchors. Predictions for new formulations are then able to be treated as perturbations from the anchored measured results to achieve higher accuracy. In one embodiment, the updated ML model, trained on both the existing data set and the new protein data set, predicts viscosity profiles for the new protein for a wide range of buffers and concentrations. In one embodiment, the pretrained ML model uses the new protein data set as context data in addition to the existing data set without fully training and modifying the pretrained ML model with the new protein data. In another embodiment, the pretrained ML model is fine-tuned with the new protein data, and then predicts viscosity profiles for a wide range of buffers and concentrations.

The user is able to select a few conditions from the predicted viscosity set for the new protein, or a selection algorithm generates suggestions for a few buffer conditions where it has the least confidence. Then, the user is then able to test viscosity of those conditions with lower confidence.

If there is a large discrepancy between the predicted viscosity values and the measured viscosity values, the new measurement data is added to the training set to further fine-tune the model, and the system generates a new predicted viscosity set. If the new measurement data is close to the predicted values (e.g., within a predetermined percentage range or confidence interval), the predictions are accurate, and the process terminates.

In one embodiment, the training set grows as a federated model and therefore becomes more accurate as more experiment data is added by a single user or by multiple users using the system. If the seed training data set does not have conditions the user is interested in, either the user can collect the desired baseline data for this specific condition and add it to the database and the training set, or can purchase more relevant data through the platform.

In one embodiment, with user consent, anonymized data is collected from users for federated learning. Efforts from multiple sites at scale improve model accuracy quicker and to a greater degree than relying on data from a single source. In one embodiment, the system includes an interface able both to request, generally, user consent to use anonymized data to train a non-local, base model, and also to receive selection of one or more parameters that the model is allowed to use or not allowed to use for training. For example, the interface is able to receive selection to allow limited information about the active compound to be used in the training data (e.g., one or more protein motifs, but not the whole compound) or limit the information to only formulation parameters such as concentration, buffer, etc., and to omit any information about the active compound itself in the training.

The invention as described herein includes both a system and a method. In one embodiment, a computer system receives an unfiltered data set, with the data set including physical measurements (e.g., from one or more viscosity tests) of one or more different formulations. The data set preferably includes information about the formulation, or different preparations of the formulation, including concentration of the active ingredient, concentration of one or more buffers, concentration of one or more surfactants, and one or more other formulation parameters.

Processors of the computer system filter the data set to create a filtered data subset, which includes removing at least one geometric parameter of the unfiltered data set, or removing variant formulations in the unfiltered data set that fall outside of predefined ranges. The filtering can include utilizing physical property models trained on selected data subsets of the unfiltered data set. The physical property models have preferably been tested for the ability to predict one or more relevant physical properties of a variant formulation with different data subsets to determine the optimal data subset to select (i.e., the filtered data subset). In one embodiment, physical property models trained with different data subsets are tested against each other as well as against a physical property model trained with the unfiltered data set to measure relative predictive power, with the target physical property to predict being the independent variable. Because data subsets having physical property information outside a relevant range for prediction are naturally less useful for training the predictive power of the model, these data subsets end up being removed. In one embodiment, genetic algorithms are used to filter out uninformative data from the unfiltered data set as part of the filtering process.

The physical property models, now trained with the filtered data subset, are able to be used for multiple purposes, including predicting relevant physical properties (e.g., viscosity) of formulations having different concentrations or other parameters, or, conversely, identifying new formulation variants predicted to have physical properties matching one or more preselected criteria.

In one embodiment, the system and method operate using a federated learning paradigm. The system receives one or more input parameters from a user that are used by an artificial intelligence algorithm to generate a formulation viscosity profile. The system can further receive one or more constraints on the formulation viscosity profile, which provide, for example, a threshold on one or more constituents of the formulation viscosity profile. Several candidate formulation viscosity profiles are able to be generated, which define a federated model. As further input parameters are provided to the federated model, a subset of the candidate formulation viscosity profiles are identified. The subset of candidate formulation viscosity profiles are able to be identified by determining that a candidate formulation viscosity profile satisfies one or more of the received constraints. For each candidate formulation viscosity profile, input data is received and a prediction is made based on the prediction data. Those candidate formulation viscosity profiles where the prediction output fails to satisfy a target performance threshold are filtered out. The system is also able to iteratively improve the input parameters used for each candidate formulation viscosity profile to better allow the profiles to satisfy the target performance threshold.

Once the system has narrowed down the candidate formulation viscosity profiles to a filtered subset (e.g., two remaining models), an optimization function is able to be used to determine which profile in the filtered subset is most preferable. The preferred profile is then used to make predictions regarding properties of new formulations or the properties of existing formulations in tests with different parameters.

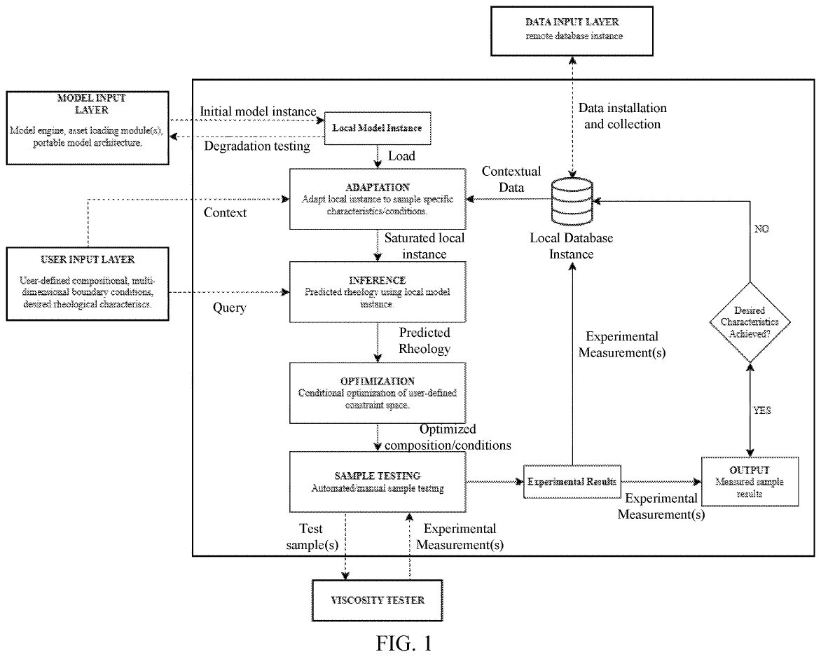

FIG. 1 is a schematic diagram of a system implementing artificial intelligence-based prediction to streamline formulation viscosity testing according to one embodiment of the invention. The system receives one or more user-defined formulization parameters and optimization space measurements. The system is able to receive these parameters and measurements either via an instance of a user-interface on a computing device (e.g., phone, computer, tablet, etc.) or via loading in one or more data files to a backend of the system. Parameters may include a chemical formula or sequence of an active compound (e.g., a protein, antibody, etc.), one or more specific motifs, functional groups, or subsequences of the active compound, a concentration of the active compound, a buffer used of the formulation, concentration of the buffer, a surfactant used in the formulation, a concentration of the surfactant, and/or one or more other parameters. For many users, it may be advantageous not to include each and every parameter of the formulation, especially the specific formula for the active compound, such as for security reasons. In one embodiment, the parameters include identification of one or more analogous compounds predicted to behave similarly to a novel active compound. The parameters may include relevant ranges for each variable, defining an optimization space for which one or more target physical properties are relevant.

In one embodiment, the system includes a user interface for inserting information regarding the formulations or active compounds to be tested. In a preferred embodiment, the user interface is able to receive different degrees and different combinations of information regarding the active compound, with more information being preferable to produce high confidence value results, but with the system still able to operate with low information for users that do not wish to divulge specific parameters of the active compound, even to a local model. In one embodiment, after input parameters for a novel formulation or active compound are provided, the platform automatically generates a prompt for additional information that was not provided that may be helpful (e.g., sequence, molecular weight, class of the protein, additional formulation parameters or ranges, etc.).

In a preferred embodiment, the received data set includes measurements for one or more target physical properties (e.g., viscosity) of formulations having different formulation parameters. In another embodiment, the received data set does not include any measurements of physical properties and makes an initial prediction solely on the structure of the active compound at target formulation parameters provided in the input data. In one embodiment, the received data set is mapped to a feature vector identifying parameters of the formulations (e.g., protein, concentration, buffer, surfactant, stabilizer, excipient, etc.) and/or parameters of the test itself (e.g., flow velocity) and maps this feature vector to the tested physical property (e.g., viscosity) as the independent variable.

In one embodiment, the system implements a local instance of a base multi-head regressor model on a user device or within a user network of multiple devices (e.g., multiple devices associated with the same company or workspace). Utilization of the local instance prevents data leakage of input data or predicted parameters of novel formulations addressed by the system. The base multi-head regressor model has been trained with numerous types of active compounds (e.g., different antibodies, different proteins) in formulations with various parameters (e.g., concentrations, buffers, pH, surfactants, etc.) with correlated physical property measurement data (e.g., experimental viscosity data) for each formulation parameter for each active compound. One of ordinary skill in the art will understand that other machine learning model types, in addition to regressor models, are also contemplated herein. In one embodiment, for example, the machine learning model utilized is a support vector machine (SVM) sequence activity model tuned specifically to identify physical properties of novel formulations based on measurement data for other formulations having one or more similar parameters. In one embodiment, a deep neural network is utilized in lieu of or in addition to the regressor model. In one embodiment, a conditional neural process is utilized in lieu of the regressor model.

In one embodiment, the machine learning model is able to automatically retrieve new, publicly available data using at least one web scraper of one or more databases of literature related to the physical property being measured.

In one embodiment, the machine learning model receives a chemical structure corresponding to a novel active compound used in a formulation, or novel buffers or surfactants used in the formulation, and is operable to automatically identify one or more functional groups, as well as the number of each identified functional group, of the provided structure to predict how the novel compound will interact with itself and with other parts of the formulation at different concentrations in order to develop the physical property profile of the formulations.

For embodiments where no measurement data has been provided, the system utilizes the local instance of the base regressor model to predict a viscosity profile within a target range of formulation parameters. For embodiments where some initial measurement data is provided, the local regressor model first makes a prediction of the target physical parameter at the tested formulation parameters and compares the prediction to the provided measurement data for the formulation with those formulation parameters to further train and update the local instance of the regressor model. The trained and updated local regressor then predicts a viscosity profile for the target range of formulation parameters.

The output of the local regressor is able to take a number of different forms. In one embodiment, the output of the local regressor includes the predicted physical properties (i.e., viscosity values) for each input formulation parameter. In one embodiment, the local regressor receives as an input a target physical property and automatically simulates different formulation parameters until the physical property is predicted to be met. For example, if a target viscosity is 15 cP, the local regressor model automatically simulates different formulation concentrations, buffers, pH, surfactants, etc. until one or more proposed formulations are provided that meet or approximate the target viscosity within a tolerance range. In one embodiment, the local regressor receives both a target physical property and a range of values for the formulation parameters that constrain the bounds of what formulations are proposed. This helps to prevent, for example, proposed formulations with too low or too high a concentration of active ingredient to be medically effective, or helps to prevent non-feasible or dangerous quantities of different components of the formulations.

In one embodiment, the output of the local regressor further includes confidence values associated with predicted physical properties for simulated formulations. The system is then able to propose one or more tests for the physical property be performed for one or more different formulations for which the confidence value is below a preset threshold. For example, if the predicted values for a given formulation are assigned a confidence value of 30%, then the system suggests that testing be done for those formulations to validate the predictions. Even where further testing is required or preferable, the amount of time, number of samples, and overall cost is greatly reduced by the system providing specifics of which tests should be performed and which are less likely to be needed.

In one embodiment, the outputs of the local regressor are suggestions and whether or not to perform the additional tests and which tests to perform are a choice for the user. In one embodiment, information regarding additional tests suggested as by the local regressor is automatically sent to one or more labs or facilities capable of performing the necessary physical testing of the formulations. In one embodiment, the computing device of the system is in communication with and able to send commands to at least one viscometer or other testing device to automatically perform the suggested tests using the proposed formulations, thereby providing a more fully automated system. In one embodiment, one or more of the suggested tests or suggested formulation variants to test are rejected by a user (e.g., as unavailable, unfeasible, etc.). If only some of multiple suggested variants or tests are rejected, then only the non-rejected/approved suggested tests or tests involved the suggested variants are performed. If all suggested variants or tests are rejected, the system is able to automatically generate new suggested tests or variants. Exemplary testing devices able to work in communication with the computing device include, but are not limited to, those described in U.S. Pat. No. 11,441,987 and U.S. Patent Pub. No. 2023/0221233, each of which is incorporated herein by reference in its entirety.

After additional testing is performed and measurement data is generated, the measurement data is provided, either automatically or manually by a user, to the system. The local regressor compares the measured data and the predicted values for the same formulations and updates the local regressor model accordingly. If the experimentation validates one or more target formulations, then the local regressor is updated for the next time the same user utilizes it for a new protein or other proposed active compound. If the experimentation shows that the testing formulations did not have target physical properties within a desired range, in contradiction of the predicted viscosity profile, then the local regressor generates a new predicted viscosity profile after being updated with the experimental data.

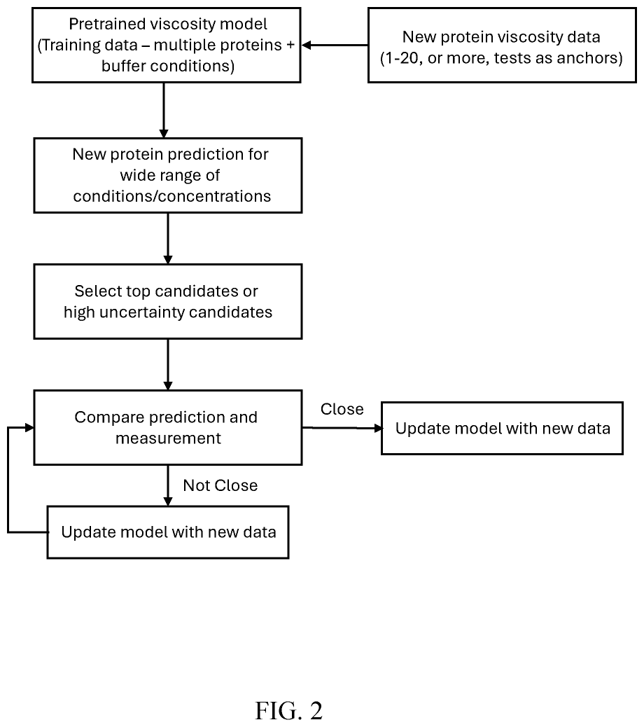

FIG. 2 is a schematic diagram of a method for training viscosity prediction models according to one embodiment of the invention. A pretrained viscosity model is implemented, trained on data including physical properties (e.g., viscosity) of multiple formulations of proteins in different buffer conditions.

The ML model adds the viscosity data generated by tests for a new protein to further train the viscosity model, with the test results for the new protein being highly weighted and acting as anchors or contextual data. Predictions for new formulations are then able to be treated as perturbations from the anchored measured results to achieve higher accuracy.

The updated ML model predicts viscosity profiles for the new protein for a wide range of buffers, pH values, and concentrations. Top candidate formulations are then selected based on which formulations are predicted to approximate one or more target physical properties. These top candidate formulations are then able to be tested in one or more measurement devices to determine if the target physical properties are indeed satisfied or if the model needs to be updated with the new measurement data and rerun predictions to identify new top candidate formulations.

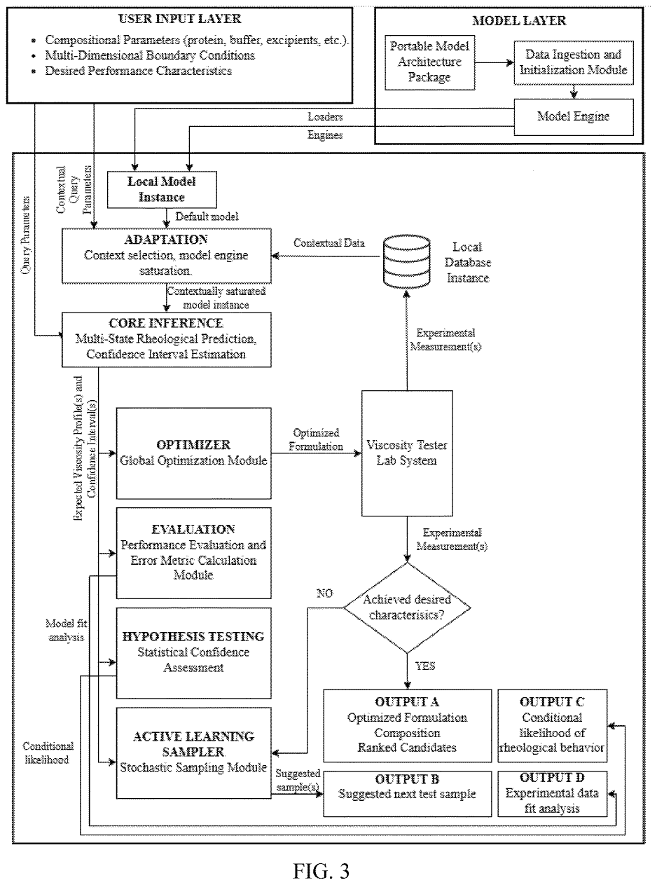

FIG. 3 is a schematic diagram for a system for predicting rheological properties of a test sample according to one embodiment of the invention. A user input layer receives input parameters and input data from a user via a user interface or through direct addition of the data to a backend of the system. The input parameters and input data include compositional parameters (e.g., protein, buffer, excipients, etc.), multi-dimensional boundary conditions, and/or desired performance characteristics for a novel formulation. A model layer also provides an initial base model with which to produce predictions, where the model layer itself performs the processes of data ingestion and initialization to train the base model before providing it to the system as a local instance. The local instance of the model can be updated based on the information provided in the user input layer, via periodic updates from the model layer, and/or via experimental data received from at least one physical property testing system.

The current version of the local instance of the machine learning model is to perform various tasks based on the data provided in the user input layer. First, the machine leaning model is able to predict an optimized formulation for achieving desired performance characteristics based on the boundary conditions and other formulation parameters provided. Second, the machine learning model is able to evaluate one or more variants of the formulation across a standardized set of conditions, or for conditions specifically designated in the user input layer. Third, the machine learning model is able to perform hypothesis testing and to provide confidence level assessments for the measurements generated. Fourth, the machine learning model is able to provide active learning sampling by providing information about what sample formulations, what tests, or what other parameters need to be tested in order for the model to be updated and to provide more accurate predictions.

In predicting an optimized formulation, the system is able to either automatically load the optimized formulation into a viscosity tester lab system (or another physical property testing system), or this choice and process is done manually by a technician based on suggestions provided by the machine learning model. Experimental measurement from the physical property testing system are provided to a local database, and then fed into the local instance of the machine learning model as contextual data to update the model for improved accuracy. If the predicted optimized formulation does have the desired characteristics, then, if there are more than formulations with the desired characteristics, the system is able to provide a ranking of optimized formulation candidates. If the predicted optimized formulation does not have the desired characteristics, then the model is updated based on the experimental data and the active learning sampler functionality of the model is able to output additional suggested tests for further improving the model.

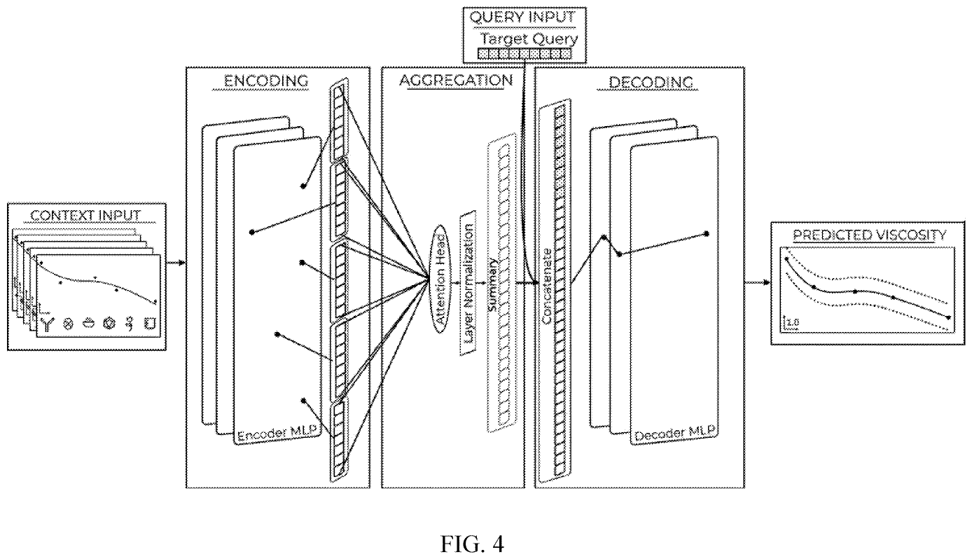

FIG. 4 is a schematic diagram for a system of generating a predicted viscosity profile based on context input and a target query according to one embodiment of the invention. In one embodiment, context input for a novel compound or formulation is injected into an encoding layer of a machine learning model (e.g., an encoder multilayer perceptron (MLP)). In one embodiment, the context input includes test data for specific physical properties (e.g., viscosity) performed for certain versions of the novel compound or formulation, or for similar compounds or formulations. The encoder layer transforms the context input into one or more feature vectors, which are then aggregated at an attention head (e.g., multi-head attention) of an aggregation layer, which weights the feature vectors and performs layer normalization across the embedded features. The aggregation layer produces a concept vector that is concatenated with a vector representing a target query in a decoding layer of the machine learning model (e.g., a decoding MLP). The concatenated vector is decoded to produce a predicted viscosity profile.

The term “viscosity” as used herein is able to refer to one or more different forms of viscosity, including but not limited to dynamic viscosity, kinematic viscosity, steady shear viscosity, relative viscosity, extensional viscosity, etc.

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A system for managing physical measurements of biologic formulations, comprising:
a computing device, including a processor and a memory, wherein the processor is configured to execute a plurality of programming instructions that, when operating on the processor, cause the computing device to:
receive input measurement data of at least one physical property of at least one variant of a biologic formulation;
update a preexisting machine learning model using the input measurement data;
utilize the updated machine learning model to generate a predicted physical property profile of the biologic formulation;
identify at least one selected variant of the biologic formulation for which additional measurement data is suggested; and
receive measurement data for at least one additional variant of the biologic formulation;
wherein, if the measurement of the at least one additional variant does not match the predicted physical property profile of the biologic formulation, generate an updated predicted physical property profile using the updated machine learning model.

2. The system of claim 1, wherein the at least one physical property includes viscosity.

3. The system of claim 1, wherein the preexisting machine learning model is a local instance of a base regressor model received from a remote server.

4. The system of claim 1, wherein the machine learning model fully updates the preexisting model by training on the new measurement data.

5. The system of claim 1, wherein the machine learning model is updated by the new measurement data as context for the biologic formulation without changing the preexisting machine learning model.

6. The system of claim 1, wherein the computing device sends commands to at least one physical property measurement system including formulation parameters of the at least one additional variant and parameters for one or more tests to be performed.

7. The system of claim 1, wherein the machine learning model is periodically updated using additional measurement data received from at least one federated server.

8. The system of claim 1, wherein the input measurement data is treated as contextual data by the trained machine learning model in generating the predicted physical property profile of the biologic formulation.

9. The system of claim 1, wherein the computing device further receives at least one target physical parameter value and determines optimal formulation parameters for a variant of the biologic formulation achieving the at least one target physical parameter value, and wherein the formulation parameters include a concentration of at least one active compound, a buffer, a pH, an excipient, a stabilizer, and/or a surfactant.

10. The system of claim 1, wherein the machine learning model generates confidence values associated with the predicted physical property profile.

11. The system of claim 10, wherein at least one selected variant is selected based on predictions of the at least one physical property for the at least one selected variant being below a predetermined confidence threshold.

12. A method of managing physical measurements of biologic formulations, comprising:
receiving input measurement data of at least one physical property of at least one variant of a biologic formulation;
updating a preexisting machine learning model using the input measurement data;
utilizing the updated machine learning model to generate a predicted physical property profile of the biologic formulation;
identifying at least one selected variant of the biologic formulation for which additional measurement data is suggested; and
receiving measurement data for the at least one additional variant of the biologic formulation;
wherein, if the measurement of the at least one additional variant does not match the predicted physical property profile of the biologic formulation, generating an updated predicted physical property profile using the updated machine learning model.

13. The method of claim 12, wherein the at least one physical property includes viscosity.

14. The method of claim 12, wherein the preexisting machine learning model is a local instance of a base regressor model received from a remote server.

15. The method of claim 12, further comprising sending commands to at least one physical property measurement system including formulation parameters of the at least one additional variant and parameters for one or more tests to be performed.

16. The method of claim 12, further comprising periodically updating the machine learning model using additional measurement data received from at least one federated server.

17. The method of claim 12, wherein the input measurement data is treated as contextual data by the trained machine learning model in generating the predicted physical property profile of the biologic formulation.

18. The method of claim 12, further comprising receiving at least one target physical parameter value and determining optimal formulation parameters for a variant of the biologic formulation achieving the at least one target physical parameter value, wherein the formulation parameters include a concentration of at least one active compound, a buffer, a pH, an excipient, a stabilizer, and/or a surfactant.

19. The method of claim 12, further comprising the machine learning model generating confidence values associated with the predicted physical property profile, wherein at least one selected variant is selected based on predictions of the at least one physical property for the at least one selected variant being below a predetermined confidence threshold.

20. A system for managing rheological testing of biologic formulations, comprising:
a computing device, including a processor and a memory, wherein the processor is configured to execute a plurality of programming instructions that, when operating on the processor, cause the computing device to:
periodically receive population training data including viscosity measurements of various biologic compounds in formulations having various parameters from a federated server to fine-tune a local instance of a machine learning model;
receive formulation parameters for a plurality of variants of a biologic formulation;
utilize the machine learning model to generate a predicted viscosity profile of the biologic formulation;
identify at least one selected variant of the biologic formulation for which additional viscosity measurement data is suggested;
receive viscosity measurement data for at least one additional variant of the biologic formulation; and
fine tune the machine learning model using the viscosity measurement data for the at least one additional variant of the biologic formulation;
wherein, if the measurement of the at least one additional variant does not match the predicted viscosity profile of the biologic formulation, generate an updated predicted viscosity profile using the updated machine learning model.
